(19) United States
(12) Patent Application Publication
(10) Pub. No.: US 2016/0148373 A1
(43) Pub. Date: May 26, 2016

(54) IMAGE RECORDING SYSTEM

(71) Applicant: TRACTUS CORPORATION, Danville, CA (US)

(72) Inventors: Bruce Alexander ROBINSON, Maple Valley, WA (US); Scott Powers HUNTLEY, Danville, CA (US)

(21) Appl. No.: 14/900,468

(22) PCT Filed: Jun. 27, 2014
(86) PCT No.: PCT/US2014/044525

§ 371 Date: Dec. 21, 2015

Related U.S. Application Data
(60) Provisional application No. 61/840,805, filed on Jun. 28, 2013.

(51) Int. Cl. G06T 7/00; A61B 8/08; A61B 8/00; G06T 7/20

(52) U.S. Cl. CPC G06T 7/0012; G06T 7/20; G06T 7/004; A61B 8/0825; A61B 8/4263; G06T 2207/10132; G06T 2207/30068; G06T 2207/30168

(57) ABSTRACT

Image recording devices and systems are disclosed along with methods for image recording. The systems can be in communication with a manual imaging device having an imaging probe configured to scan a volume of tissue and output scan images. The systems can be further configured to electronically receive first and second images and to calculate an image-to-image spacing between the first and second images. The systems can further perform an image quality analysis on the scan images and record the scan images if movement of the imaging probe is detected and the scan images satisfy the image quality analysis. The systems can also include a position tracking system. Position sensors and/or orientation sensors can be coupled to the imaging probe to determine the position and orientation of the imaging probe. The systems can be configured to associate the position and orientation data with the scanned images.

### CROSS REFERENCE TO RELATED APPLICATIONS

This application claims the benefit of U.S. Patent Appl. No. 61/840,805, filed Jun. 28, 2013, the disclosure of which is incorporated herein by reference.

### INCORPORATION BY REFERENCE

All publications and patent applications mentioned in this specification are herein incorporated by reference to the same extent as if each individual publication or patent application was specifically and individually indicated to be incorporated by reference.

### FIELD

Embodiments described herein relate to systems and methods for image recording.

### BACKGROUND

Recording images using a hand-held imaging probe requires hand-eye coordination as well as the ability to control several functions at once. If the images are recorded in a temporal manner, that is a constant number of frames per second, then the number of sequential images recorded is a function of how long the recording function is “on”. Constant recording may not be desirable because each image recorded requires computer memory resources and operator time to review.

By the way of example, if the record function is “on”, but the probe is not contacting tissue, then the images recorded have no information. The system would waste computer storage resources to store these “empty” images and waste the reviewer's time to review these empty images.

By the way of another example, if the probe is on the tissue then images with viable information would be recorded, but if the probe does not move then the same information would be available in multiple images. The system would waste computer storage resources to store these “identical” images and waste the reviewer's time to review these identical images.

One method of solving this problem would be to consciously activate the recording function when the user wants images recorded and consciously deactivate the recording function when the user does not want the images recorded. Thus, the user could turn the recording function “on” when the user judges that the device has an image with information that is worthy of recording and when the imaging probe moves and the sequence of images changes such that the images recorded are unique. That conscious “on” and “off” feature could be a manually activated button, a foot pedal, or voice activation (for example, saying the word “on” to turn the recording on and saying the word “off” to turn the recording off).

This method is cumbersome, often impossible. When scanning with a hand-held probe it is often necessary to use one hand to manipulate the probe and a second hand to stabilize the tissue to be scanned. This procedure does not allow a free hand to manipulate the “on” and “off” procedure. In addition, when scanning the tissue the user must often focus their vision on the tissue to be scanned and averting that visualization to find an “on” or “off” button to press. The same challenge that applies to visualizing manual buttons applies to finding a foot pedal to depress. Additionally, with audible commands, these can be distracting to the patient.

As such, embodiments described herein relate to systems and methods to automate or control the start and stop of image recording by an imaging device based on image device movement and/or image quality of recorded images.

### SUMMARY OF THE DISCLOSURE

Methods, apparatus, and systems for use with an ultrasound imaging console in screening a volume of tissue are disclosed herein. The targeted human tissue can include a human breast.

In general, in one embodiment, a tissue imaging system includes an image recording system in communication with a manual imaging device having an imaging probe. The manual imaging device is configured to scan a volume of tissue and output a first scan image and a second scan image. The image recording system is configured to electronically receive the first and second scan images, calculate an image-to-image spacing between the first and second scan images, determine whether the image-to-image spacing indicates movement by the imaging probe, determine an image quality of the first and second scan images if the image-to-image spacing indicates movement, and record the first and second scan images where the calculated image-to-image spacing indicates movement by the imaging probe and the image quality analysis indicates that the first and second scan images satisfy a pre-determined image quality. A position tracking system is configured to detect and track the position of the imaging probe and provide location identifier information for the first and second scan images. The position tracking system can be configured to electronically output probe position data and the location identifier information to the image recording system.

This and other embodiments can include one or more of the following features. The image recording system can be configured to store a user-defined image-to-image distance limit for comparison to the calculated image-to-image spacing and automatically record the first and second scan images if the calculated image-to-image spacing is equal to or more than the user-defined image-to-image distance limit and if the scan images satisfy the pre-determined image quality. The user defined image-to-image spacing can be about 1 mm or less. The system can further include electromagnetic position sensors. The system can further include magnetic position sensors. The system can further include microwave position sensors. The system can further include position sensors that are optical markers imaged by a plurality of cameras. The position sensors can be infrared markers imaged by a plurality of cameras. The position sensors can be ultraviolet markers imaged by a plurality of cameras. The image quality can be determined based on a percentage of tissue imaged information present in the scan image. The pre-determined image quality can be tissue imaged information present in the scan image of greater than about 50%. The percentage can be a user-defined value corresponding to the variation in gray-scale within an image area of neighboring pixels. The user-defined value for the variation in the gray-scale within the image area can correspond to pixel value differences of less than 16 values on a 256 level gray scale. The system can be configured to only record images that indicate movement of the imaging probe and satisfy the image quality analysis. The system can further include orientation sensors.

In general, in one embodiment, a method of recording images of a tissue structure includes: (1) electronically receiving a first scan image generated from an imaging device; (2) electronically receiving a second scan image generated from an imaging device; (3) calculating an image-to-image spacing between the first and second scan images based on position data received from a plurality of sensors coupled to the imaging device; (4) comparing the calculated image-to-image spacing to a stored predetermined distance value; (5) performing an image quality analysis on the first and second scan images if the image-to-image spacing exceeds the stored predetermined distance value; (6) recording the first scan image if the first scan image satisfies the image quality analysis; and (7) recording the second scan image if the second scan image satisfies the image quality analysis.

This and other embodiments can include one or more of the following features. The image quality analysis can be a pixel color analysis. The pixel color analysis can include grouping together neighboring pixels that are within 16 values on a 256 level gray scale of a user-defined value. The processor can be configured to divide the first and second scan images into segments and compute the pixel values for each segment for image quality analysis. The imaging device can be an ultrasound probe. The sensors can include position sensors. The sensors can include orientation sensors. The first and second scan images can be only recorded if the image-to-image spacing exceeds the predetermined distance value and the scan images satisfy the image quality analysis. The image quality can be determined based on a percentage of tissue imaged information present in the scan image. The pre-determined image quality can be tissue imaged information present in the scan image of greater than about 50%. The user defined image-to-image spacing can be about 1 mm or less.

In general, in one embodiment, a method of recording images of a tissue structure includes: (1) electronically receiving a position data for an imaging probe from position and/or orientation sensors coupled to the probe to detect movement of the imaging probe; (2) electronically receiving a first scan image generated from the imaging probe; (3) performing an image quality analysis on the first scan image if movement of the imaging probe is detected; and (4) recording the first image if the first scan image satisfies the image quality analysis and movement of the imaging probe is detected.

This and other embodiments can include one or more of the following features. The movement of the probe can be detected based on the position and/or orientation sensors coupled to the probe. Movement can be determined based on an image-to-image spacing computed by a processor and compared to a predetermined distance value stored in the processor. The predetermined distance value can be about 1 mm or less. The image quality can be determined based on a pre-determined percentage of tissue imaged information present in the scan image. The pre-determined image quality can be tissue imaged information present in the scan image of greater than about 50%. The image quality analysis can be a pixel color analysis. The pixel color analysis can include grouping together neighboring pixels that are within 16 values on a 256 level gray scale of a user-defined value.

In general, in one embodiment, a tissue imaging system includes an image recording system in communication with a manual imaging device having an imaging probe. The manual imaging device is configured to scan a volume of tissue and output a first scan image and a second scan image. The image recording system is configured to electronically receive the first and second scan images, calculate an image-to-image spacing between the first and second scan images, determine whether the image-to-image spacing indicates movement by the imaging probe, determine an image quality of the first and second scan images if the image-to-image spacing indicates movement, and automatically record the first and second scan images where the calculated image-to-image spacing indicates movement by the imaging probe and the image quality analysis indicates that the first and second scan images satisfy a pre-determined image quality. A position tracking system is configured to detect and track only the position or position and orientation of the imaging probe and provide location identifier information for the first and second scan images. The position tracking system can be configured to electronically output probe position data and the location identifier information to the image recording system.

This and other embodiments can include one or more of the following features. The system can further include position sensors or position and orientation sensors. The image recording system can be configured to store a user-defined image-to-image distance limit for comparison to the calculated image-to-image spacing and automatically record the first and second scan images if the calculated image-to-image spacing is equal to or more than the user-defined image-to-image distance limit. The user defined image-to-image spacing can be about 1 mm or less. The image quality can be determined based on a percentage of tissue imaged information present in the scan image. The pre-determined image quality can be tissue imaged information present in the scan image of greater than about 50%. The percentage can be a user-defined value corresponding to the variation in gray-scale within an image area of neighboring pixels. The user-defined value for the variation in the gray-scale within the image area of neighboring pixels can correspond to pixel value differences of less than 16 values on a 256 level gray scale. The system can be configured to only record images that indicate movement and satisfy the image quality analysis.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-B illustrate images of tissue volumes. FIG. 1B shows partial shadowing due to limited contact.

FIGS. 2A-B illustrate two ultrasound images with varying degrees of shadowing.

FIGS. 3A-B show two images with segmentation.

FIGS. 4A-B show two images with usable vs. unusable information.

FIGS. 5A-C show analysis of subsequent image segments.

FIGS. 6A-D show an image recording session where movement and image quality stop or start recording.

FIGS. 7A-D show an image recording session where movement and image quality stop or start recording.

FIGS. 8A-D show an image recording session where movement and image quality stop or start recording.

FIGS. 9A-D show an image recording session where movement and image quality stop or start recording.

FIGS. 10A-D show an image recording session where movement and image quality stop or start recording.

FIG. 11 is a flow chart of an exemplary recording process.

FIG. 12 is a pixel array.

FIG. 13 is an exemplary tissue imaging system with an image recording system, position tracking system, and an imaging system.

FIG. 14 illustrates discrete images in a scan sequence.

FIG. 15 illustrates a cross-sectional view of a breast scanned by an ultrasound probe.

FIG. 16 illustrates a pixel density in a scan sequence.

FIG. 17 illustrates pixels from an image.

FIGS. 18A-D illustrates various images with differing grayscale sections.

### DETAILED DESCRIPTION

Embodiments described employ the use of image analysis and position sensors or a combination of position sensors and orientation sensors to measure whether the system is producing an image worth recording and whether the probe is being moved (e.g., creating different or unique images).

### Recording Based on Image Quality

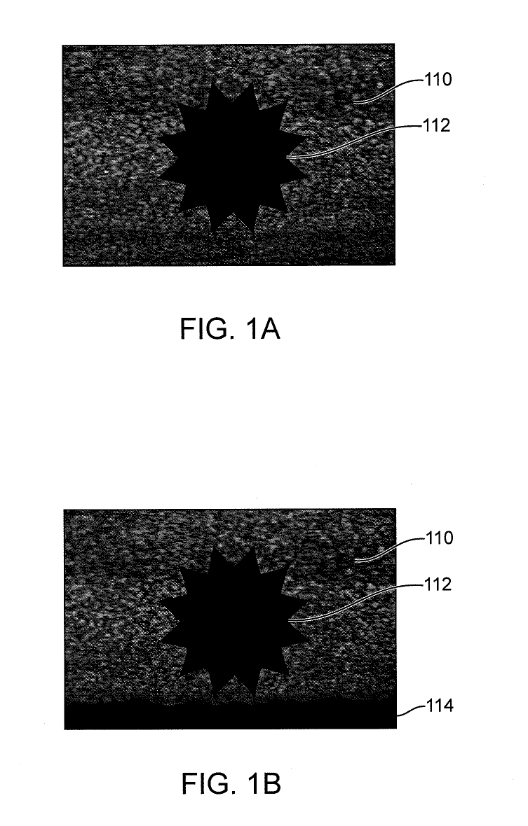

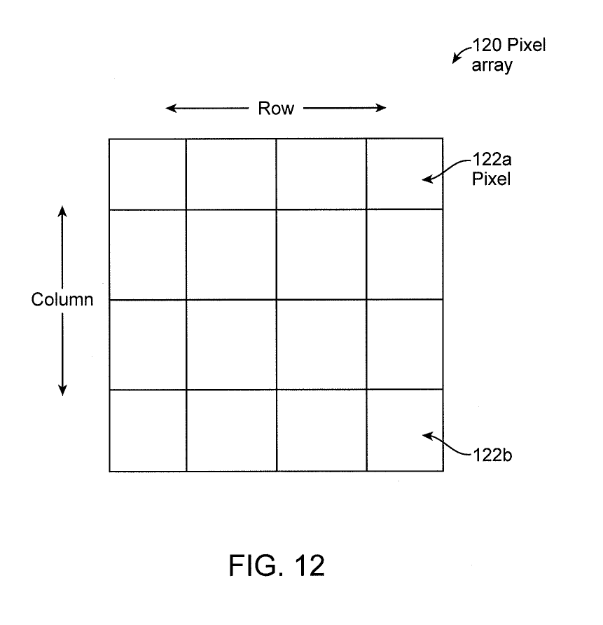

In the example of an ultrasound image, the image itself is an array of pixels (see FIG. 12). Each pixel 122 represents the acoustic reflection properties of a portion of the tissue. The pixels tend to be various shades of white to black, including greys. If the user does not apply the probe correctly then the acoustic propagation may lose integrity and part of the image may show no reflective properties. As shown in FIG. 1B, in the case where the reflective properties are completely compromised, that portion of the image 114 will not have a collection of white-to-black pixels (including greys) 110, but will be almost entirely black. FIG. 1B shows partial shadowing because of limited contact between the tissue and the probe. In this case, 10% of the image is compromised. As such, significant information is still available.

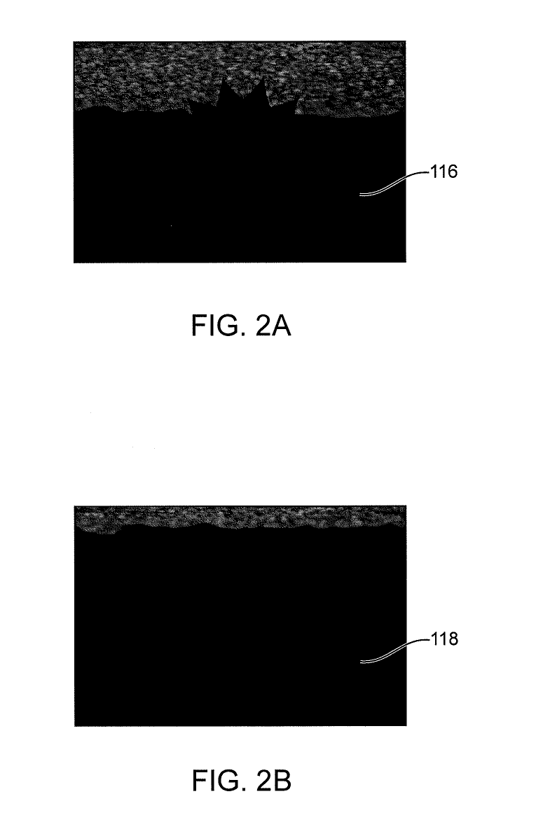

In the example of an ultrasound image, the image itself is an array of pixels. Each pixel represents the acoustic reflection properties of a portion of the tissue. If the user does not apply the probe correctly then the acoustic propagation may lose integrity and part of the image may show no reflective properties. An entire portion 116 of the image may be black (FIG. 2A). If there is no patient contact then there may be no acoustic reflection, or only minor acoustic reflection as a function of a thin layer of residual acoustic gel that may be on the end of the probe (FIG. 2B). FIG. 2A shows significant shadowing 116 because of limited contact. In this case 67% of the image is compromised. Partial information still available. FIG. 2B shows near-complete shadowing 118 because of limited contact or no contact. Partial image may be due to layer of ultrasound gel on the end of the probe. In this case 90% of the image is compromised. Most information is lost.

Some embodiments contemplated provide for image recording systems and methods that use computer programming logic to analyze portions of the image to determine whether to record that image. For example, if a section of neighboring pixels of the image are determined to be black. The system may compute the percentage of black area. This percentage can be compared with a preset acceptable value. If the computed percentage is within acceptable limits, the system will record the image. Otherwise, the image is not recorded. If the user selects a value, for example, 67% of the segments being essentially black, to label an image as having no information, then criteria may be established to determine which images to record and which to ignore. In some embodiments the percentage of tissue image in the scanned image is predetermined by the user for the image quality analysis. In some embodiments the image quality analysis preset value is about 25% tissue image or greater. In some embodiments the image quality analysis preset value is about 33% tissue image or greater. In some embodiments the image quality analysis preset value is about 50% tissue image or greater. In some embodiments the image quality analysis preset value is about 66% tissue image or greater. In some embodiments the image quality analysis preset value is about 75% tissue image or greater.

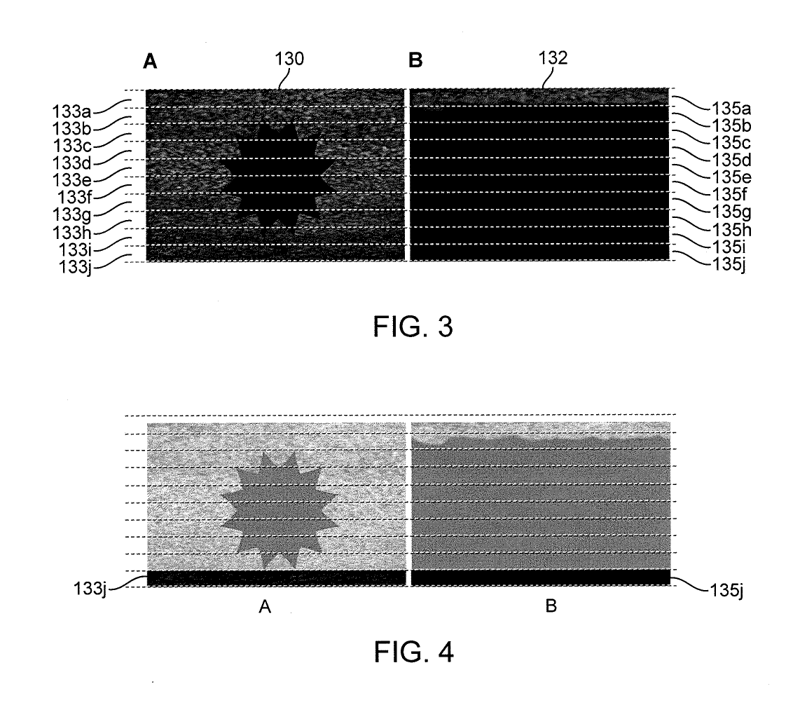

One or more image segments of an image may be analyzed to determine the percentage of usable or unusable information in the image. As shown in FIGS. 3A-B, in some embodiments once an image is received, the image is segmented for analysis. FIG. 3A shows an image 130 with usable information. FIG. 3B shows an image 132 with sections without usable information. Both images have been segmented into sections (e.g. 133a-j and 135a-j) for analysis.

In some embodiments, see FIGS. 4A-B, image segments are analyzed to determine if there are a variety of pixel colors, representing possible usable image information (FIG. 4A) or, essentially one color, representing no usable image information (FIG. 4B). In some variations, determining whether an area of an image is essentially one color is also a user-defined issue. In most electronic image presentation devices a single pixel may be represented in one of 256 shades of grey. Tissue is heterogeneous, so it is unlikely that a majority of pixels within a given area will have the same pixel color. In the way of example, if 50% of the pixels are white, are black, or even the exact same shade of grey, the image does not represent actual tissue. Conversely, if the probe is not touching tissue and the image represents a probe in air, or water, or any other homogeneous media, it is possible for 100% of the pixels to be the exact same color.

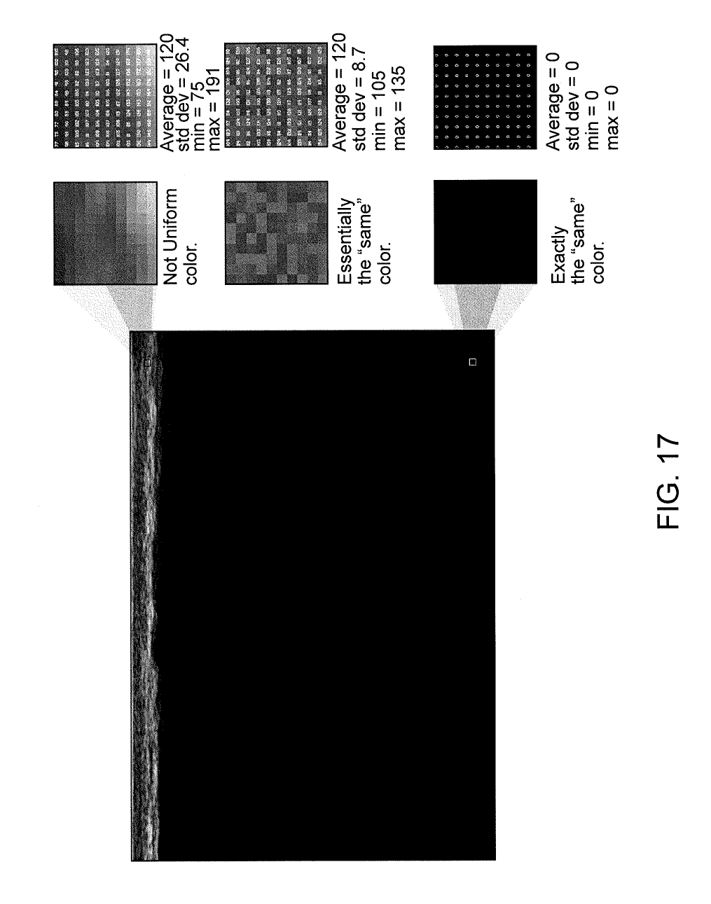

Additionally, the term “same color” or substantially the same color does not require the pixel color to be the exact same color. For example, two pixels may not have identical levels of gray. Rather, these terms may include a range in color difference between pixels that still fall into the “same” category. As a non-limiting example, pixel value differences less than 16 values on a 256 level gray scale may be considered the same color or substantially the same in color. As can be seen in FIG. 17, the individual pixels of an ultrasound image of tissue typically has a wide range of gray values, presenting the image of tissue patterns. When the pixels are uniform, or relatively uniform in color, it indicates that the ultrasound reflections are not describing tissue. The top right section of the scan illustrates a non-uniform image with multiple colors. The top right section of the scan has an average pixel value of 120 with a pixel value standard deviation of 26.4. The right middle section of the scan illustrates a section with essentially the “same” color with an average pixel value of 120 and a pixel value standard deviation of 8.7. The right bottom section illustrates a section with exactly the same color with an average pixel value of 0 and a pixel value standard deviation of 0.

As such, in some variations, embodiments described herein analyze the color of segments, neighboring pixels, or sections of an image to determine if a certain percentage of the segment is of the same or similar color. Referring to FIG. 4A, segment 133j shows a variation in shades of gray that correlates to a heterogeneous tissue substrate. In contrast, segment 135j of FIG. 4B has a uniform black color, which indicates a homogeneous medium has been imaged, which likely is not the target tissue.

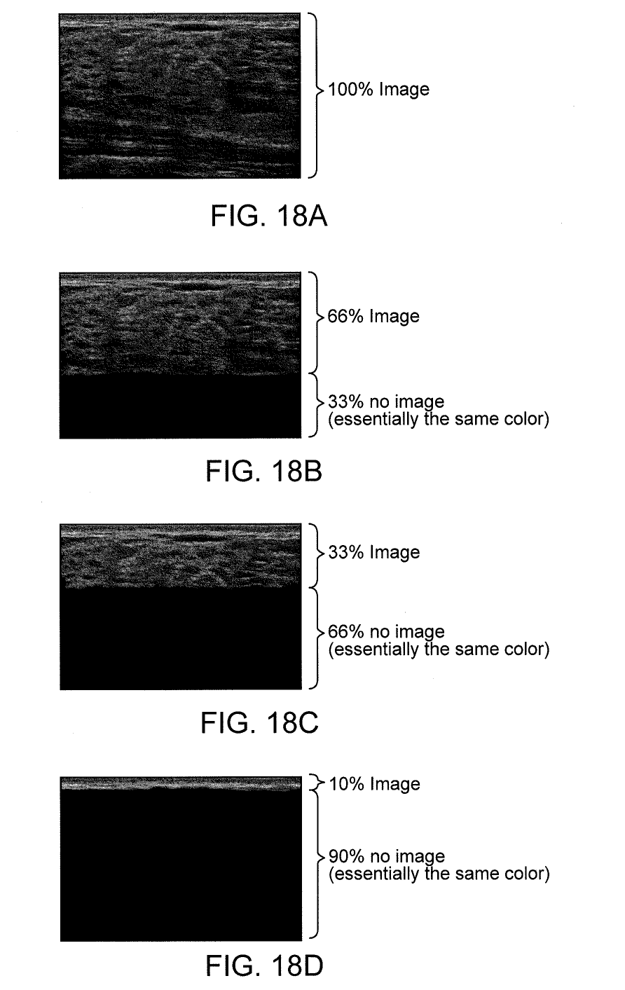

To segment or divide the images, some embodiments will apportion the image into three sections. In some cases, the sections are a left third, middle third, and a right third. In some cases, the sections are a top third, middle third, and a right third. In some embodiments, the middle third does not include a quarter of the image on each side. FIGS. 18A-D provide examples of figures having monochrome sections. The top image illustrates a scan image with 100% tissue image. The scan image second from the top illustrates a scan image with 66% tissue image and 33% no image. The scan image second from the bottom illustrates a scan image with 33% tissue image and 66% no image. The scan image on the bottom illustrates a scan image with 10% tissue image and 90% no image.

Embodiments contemplated include image recording systems and methods that analyze the color scheme and patterns in one or more segments of an image to determine, at least, whether there is a portion of the image that does not contain target tissue information and what percentage of the image contains target tissue information. In some embodiments, if a preset value or percentage of the image does not contain tissue information, then the image will not be recorded. This quality control feature improves adequate imaging of tissue and efficient image storage.

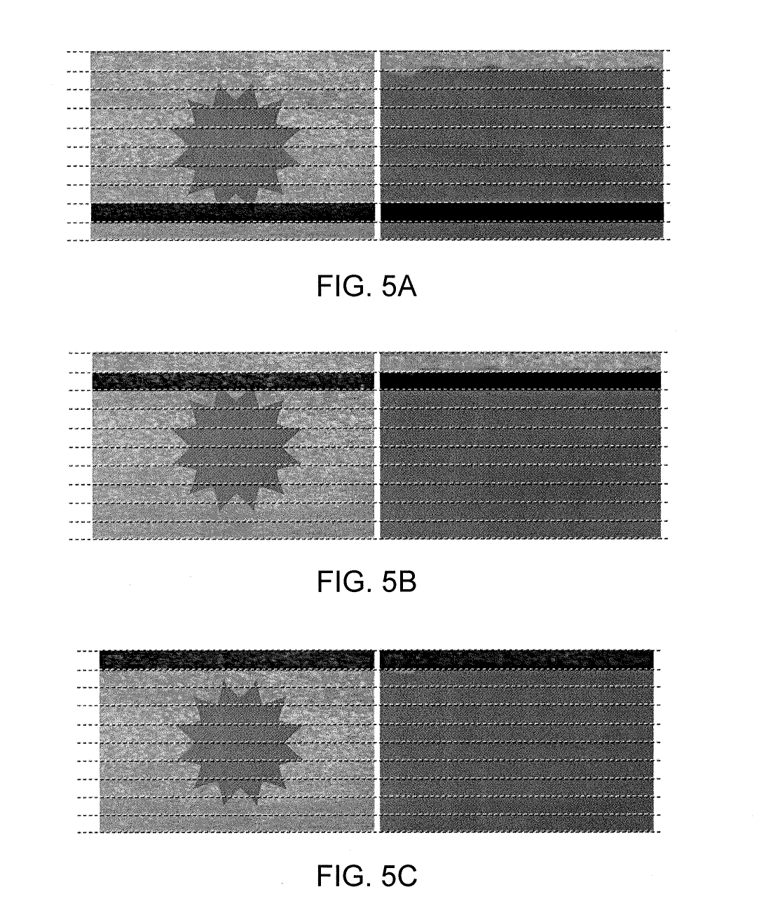

As shown in FIGS. 5A-C, subsequent image segments may also be analyzed to determine if there are a variety of pixel colors, representing possible usable image information (left on FIGS. 5A, 5B, and 5C; as well as right on 5C) or, essentially one color, representing no usable image information (right on 5A and 5B). As shown, in some images, the majority or a large portion of the image may contain substantially the same color, which would indicate that a large portion of the image likely does not contain information regarding the target structure. In other cases, a small section of the image may have unusable information while a majority of the image provides relevant information.

In some variations, the image analysis includes the step of subtracting a subtraction “black” value from each pixel. The “subtraction black” value is a background that is indicative of no tissue reflection, and may not be “black”. For example, a full-black value may have a value between about 10-20, while a uniform background, not representing tissue, may have a value between 105 and 135 (see FIG. 17) and not look “black”. That background value may be subtracted from all pixels to make the uniform areas black, and the pixels representing actual tissue more descriptive. The ability of distinguishing non-tissue pixels from tissue pixels is then enhanced. The subtraction black value may be auto-tuned at run time. Then the average sample value for each segmented portion of the image is computed. If any of the segmented values are above a cutoff, then the image can be recorded. If not, the image is not recorded and deleted.

Additionally, to analyze the image, some embodiments modify the image before analysis. For example, in the case of ultrasound, these images may include a section, such as a frame or band that outlines the image, which does not contain tissue information. As such, some embodiments include the step of removing or disregarding the frame or band prior to image analysis. In some cases, the top 15% of an image is removed or disregarded before pixel analysis.

Once it is determined that the image has sufficient usable information, the image recording systems records the image. In some embodiments, the presence of a satisfactory amount of usable information in a received image will activate the recording mode of the automatic image recording system. For example, the recording starts when the analyzed image has less than “X” percentage of a monochromatic color (e.g. black).

### Recording Based on Movement

In additional embodiments, the image recording system may record an image based on the movement of an imaging device. In some embodiments, the movement of the imaging device may be used alone or in combination with the pixel analysis described to control whether the image recording system records or deletes a received image.

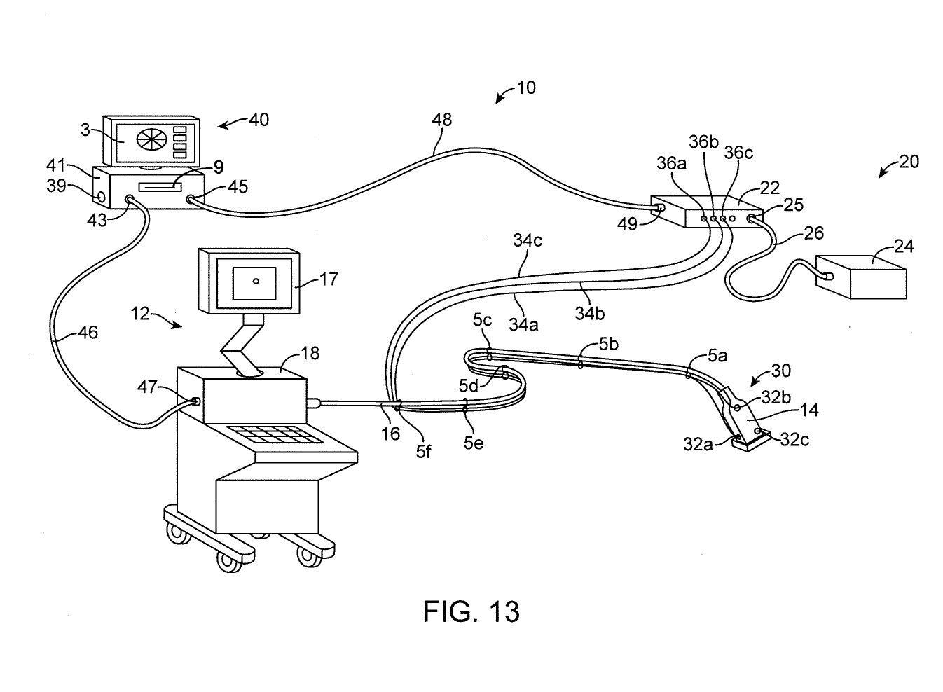

Movement of an imaging device, such as the movement of a manual imaging probe for an ultrasound device, can be measured by detecting the location of the imaging device. Systems, devices, and methods for detecting the location of an imaging device are described in detail in U.S. patent application Ser. Nos.: 13/854,800 filed on Apr. 1, 2013; 61/753,832 filed Jan. 17, 2013; and 61/817,736 filed on Apr. 30, 2013, which are incorporated by reference in their entirety. For example, FIG. 13 shows a non-limiting exemplary system with position and/or orientation sensors that can be used to detect location and movement of an imaging probe. FIG. 13 illustrates two subsystems. A first subsystem is the hand-held imaging system 12, which includes hand-held imaging monitor console 18, display 17, hand-held imaging probe 14 and connecting cable 16. A second system (referred to hereinafter as the “Image Recording System”), according to the invention, is represented in general at 10. The Image Recording System 10 comprises a data acquisition and display module/controller 40 including microcomputer/storage/DVD ROM recording unit 41, and display 3.

The Image Recording System 10 also comprises position-tracking system 20, which includes, by way of example, position tracking module 22 and position sensor locator, such as a magnetic field transmitter 24. In addition, the Image Recording System 10 also comprises a plurality of position sensors 32a, 32b and 32c coupled or affixed to the hand-held imaging probe 14. Although the hand-held imaging system 12 is shown as a subsystem separate from the scanning completeness auditing system 10, in some embodiments, the two systems are part of the same overall system. In some cases, the imaging device may be part of the scanning completeness auditing system.

Still referring to FIG. 13, hand-held imaging system 12 is connected to data acquisition and display module/controller 40 via data transmission cable 46 to enable each frame of imaging data (typically containing about 10 million pixels per frame) to be received by the microcomputer/storage/DVD ROM recording unit 41 the frequency of which is a function of the recording capabilities of the microcomputer/storage/DVD ROM recording unit 41 and the image data transmission capabilities, whether it is raw image data or video output of the processed image data, of the hand-held imaging system 12. Position information from the plurality of position sensors 32a, 32b, and 32c, is transmitted to the data acquisition and display module/controller 40 via the transmission cable 48. Cable 46 is removably attached to microcomputer/storage/DVD ROM recording unit 41 of data acquisition and display module/controller 40 with removably attachable connector 43 and is removably connected to diagnostic ultrasound system 12 with connector 47.

The position tracking module 22 is connected to data acquisition and display module/controller 40 via data transmission cable 48 wherein cable 48 is removably attached to microcomputer/storage/DVD ROM recording unit 41 of data acquisition and display module/control 40 with connector 45 and is removably connected to position tracking module with connector 49. Position sensor locator, such as a magnetic field transmitter 24 is connected to position tracking module 22 via cable 26 with removably attachable connector 25. Hand-held imaging probe assembly 30 includes, by way of example, position sensors 32a-32c, which are affixed to hand-held imaging probe 14 and communicate position data to position tracking module 22 via leads 34a-34c, respectively, and removably attachable connectors 36a-36c, respectively. Position sensor cables 34a-34c may be removably attached to ultrasound system cable 16 using cable support clamps 5a-5f at multiple locations as seen in FIG. 13.

Any suitable sensor may be used to provide location and position data. For example, magnetic sensors, optical markers (e.g. to be imaged by cameras), infrared markers, and ultraviolet sensors are examples of suitable options. Furthermore, a position sensor may or may not be a separate sensor added to the imaging device. In some cases, the sensor is a geometric or landmark feature of the imaging device, for example, the corners of the probe. In some embodiments, the optical, infrared, or ultraviolet cameras could capture an image of the probe and interpret the landmark feature as a unique position on the imaging device.

Although certain location and motion recognition methods have been described (e.g. FIG. 13), it can be appreciated that any location and motion recognition methods, software, devices, or systems can be used with the described embodiments. For example, sonar, radar, microwave, or any motion or location detection means and sensors may be employed.

Moreover, in some embodiments, sensors may not need to be added to the imaging device. Rather, location and motion detection systems can be used to track the position of the imaging device by using geometric or landmark features of the imaging device. For example, a location system may track the corners or edges of an ultrasound imaging probe while it is scanned across a target tissue.

Additionally, the sensors may also provide orientation data such as pitch, roll, and yaw. Such sensors may be position and/or orientation sensors that detect either position and/or orientation data. In some cases, a position sensor may only detect position. The system may then derive the undetected orientation information if needed. In other embodiments, the sensors may detect both position and orientation.

In some embodiments, movement of an imaging device may activate the recording system. For example, the recording system activates recording mode after detecting specific patterns of movements such as shaking, rotating, or tapping an imaging probe. The movement may be detected by a position tracking system and communicated to a recording system that activates when a specific movement is detected. In some variations, a recording system uses both movement of the imaging device and image analysis to activate recording function. For example, the recording system begins to receive images from an imaging probe/device/system once an activation motion is detected. The recording system then performs pixel analysis of the images to determine whether to record the images.

In further embodiments, the recording of an image may also be dependent on image-to-image spacing between two sequential images. The user can set a spatial limit on the image-to-image spacing which represents “movement”. For example, if two sequential images are spaced more than 0.01 mm, more than 0.1 mm apart, or more than 1 mm apart, or any other user-defined limit, the probe would be deemed as “moving” and recording may be possible.

Image-to-image spacing may be determined by any appropriate method or system including those described in U.S. patent application Ser. Nos.: 13/854,800 filed on Apr. 1, 2013; 61/753,832 filed Jan. 17, 2013; and 61/817,736 filed on Apr. 30, 2013, which are incorporated by reference in their entirety. In some embodiments, measuring or calculating the spacing or distance between individual images in a scan sequence may be referred to as determining the image-to-image resolution or spacing between discrete images in a scan sequence. Alternatively, frame to frame resolution may also be used to describe the spacing/distance between images in a scan sequence. As described, if the calculated space or distance between individual images satisfies a minimum or maximum value, the image recording system may then record these images. Additionally, the image recording system may also perform image analysis such as pixel color analysis to determine if a particular image has sufficient usable information warranting recording.

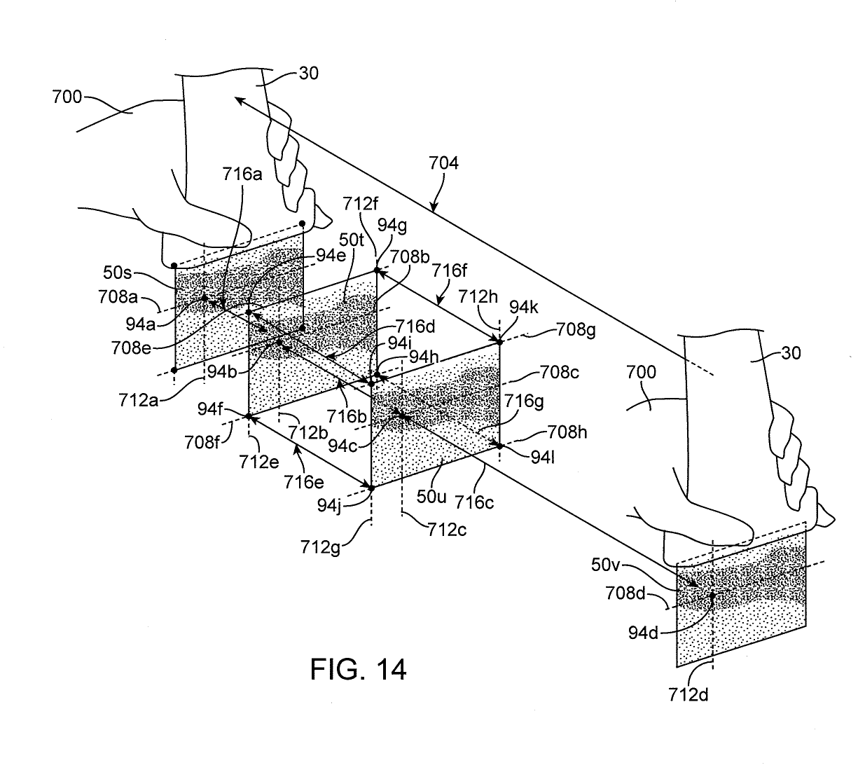

FIGS. 14-16 are related to exemplary methods and systems for computing image-to-image spacing in a scan sequence. By way of example and referring first to FIGS. 13-14, the hand-held ultrasound probe assembly 30 is translated across the surface of the skin by the human hand 700. That translation will follow a linear or non-linear path 704, and there are a series of corresponding ultrasound beam positions 50s-50v, each with a corresponding ultrasound image that is received by an image recording system 10. In particular, ultrasound image may be communicated to the acquisition and display module/controller 40 via the data transmission cable 46, to be received by the microcomputer/storage/DVD ROM recording unit 41, the frequency of which is a function of the recording capabilities of the microcomputer/storage/DVD ROM recording unit 41 and the image data transmission capabilities.

Again referring to FIG. 14, the images are stored as a set of pixels, including pixels 94a-94l, which are displayed in a two-dimension matrix of pixels, each matrix consisting of horizontal rows 708a-708h and vertical columns 712a-712h. A single pixel 94a-94h, is displayed has a unique display address P(rₓ, cₓ), where rₓ is the row of pixels on the image, r₁ being the row at the top, e.g. 708e, or the row representing structures closest to the probe, and nag being the row at the bottom (e.g. 708f), or the row representing structures furthest away from the probe; and where cₓ is the column of pixels on the image, c₁ being the column on the left (as viewed by the reviewer, e.g. 712g), and cₗₐₛₜ being the column on the right (as viewed by the reviewer, e.g. 712h). A typical ultrasound image will have between 300 and 600 horizontal rows 708 and between 400 and 800 vertical columns 712. Thus, a typical ultrasound image shall have between 120,000 and 480,000 pixels 94.

Referring again to FIG. 14, the image for each ultrasound beam position 50s-50v will have an identical pixel format. A corresponding row is the row 708 which is displayed at the same distance, vertical from the top, in every image. The depth, as measured as distance away from probe, shall be the same for corresponding horizontal rows 708. In the way of example, the information in the 8^{th} horizontal row 708 in one image represents structures which are the same distance, away from the probe at the time they are generated, as the location of the information in the 8^{th} horizontal row 708 in another image at the time that image is generated. The same logic applies to the corresponding vertical columns 712. By way of example, the information in the 12^{th} vertical column 712 in one image represents structures that are the same distance, horizontally, from the center of the probe at the time that image is recorded as the location of the information in the 12^{th} vertical column 712 in another image at the time it is recorded. Thus, the information described any one pixel 94, P(rₓ, cₓ), in one image is the same distance away from the surface of the probe (depth) and from the center line of the probe as the information described at the same pixel 94 location P(rₓ, cₓ), in another image. These pixels 94 that share common locations on the image format for the discrete images in the image sets are termed corresponding pixels 94.

One embodiment to determine the distance between images is to calculate the maximum distance between any two adjacent image frames. Since the frames are planar, then the largest distance between any two frames will occur at the corresponding pixels 94 that are at one of the four corners. Thus, the maximum distance 716 between any two corresponding frames shall be:

{Maximum Distance between any Two Corresponding Frames}=

=MAX(DISTANCE(P(FIRST-ROW, FIRST-COLUMN)−P′(FIRST-ROW, FIRST-COLUMN)),

DISTANCE(P(FIRST-ROW, LAST-COLUMN)−P′(FIRST-ROW, LAST-COLUMN)),

DISTANCE(P(LAST-ROW, FIRST-COLUMN)−P′(LAST-ROW, FIRST-COLUMN)),

DISTANCE(P(LAST-ROW, LAST-COLUMN)−P′(LAST-ROW, LAST-COLUMN)))

Where P and P′ are the corresponding pixels 94 in two adjacent images, MAX is the maximum function which chooses the largest of the numbers in the set (in this example 4) and DISTANCE is the absolute distance 716 between the corresponding pixels.

In other embodiments, the relative distance between two, adjacent, images is measured by calculating the maximum of the distances between each of the four corners of the images ({x₀,y₀,z₀}−{x₀′,y₀′,z₀′}−{x₁′,y₁′,z₁′}, {x₂,y₂,z₂}−{x₂′,y₂′,z₂′}, {x₃,y₃,z₃}−{x₃′,y₃′,z₃′}). These distances may be found via the method of Pythagoras where:

{x₀,y₀,z₀}−{x₀′,y₀′,z₀′}=sqrt ({x₀−xₒ′}²+{y₀−y₀′}²+{z₀−z₀′−z₀′}²)

Exemplary distances are shown in FIG. 14 at 716a between pixel 94a and corresponding pixel 94b; 716b between pixels 94b and 94c; 716c between 94c and 94d; 716d between 94e and 94i; 716e between 94f and 94i; 716f between 94g and 94k; and 716g between 94i and 94l. This method of measuring image-to-image spacing allows the image recording system to detect when the imaging device is moving, for example, across a tissue volume. If the distance between pixels satisfies an acceptable spacing/distance then the image recording system may activate image analysis and/or image recording activity.

In some cases, the acceptable spacing/distance is a preselected or predetermined value. In some cases, the value is a user defined limit. In other embodiments, the system may provide a range or acceptable spacing/distances for selection based on the type of exam or characteristics of the patient or target region for scanning.

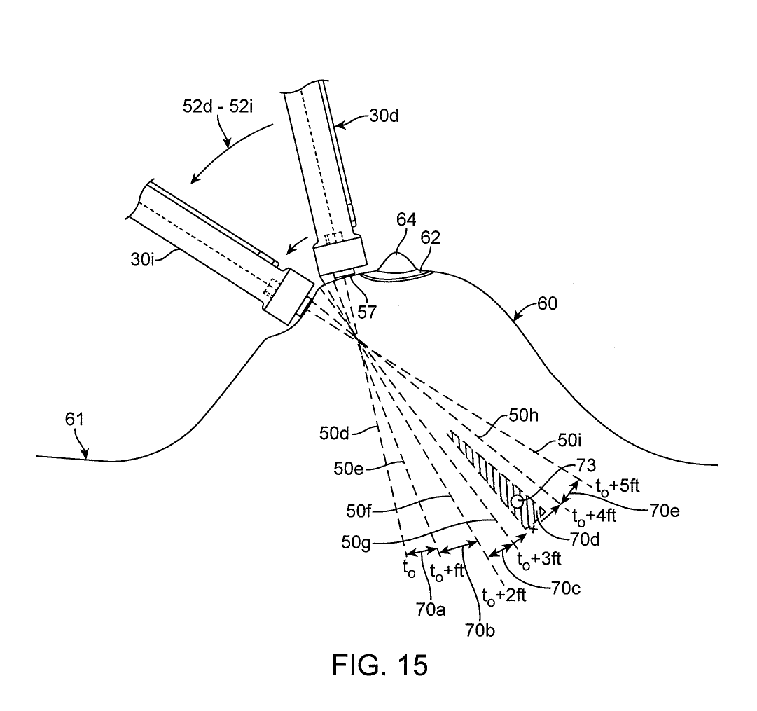

FIG. 15 provides another method of assessing frame-to-frame or image-to-image spacing. FIG. 15 shows the hand-held ultrasound probe assembly 30 at two adjacent positions 30d and 30i. For this example, assume that the rate of producing new ultrasound images is accomplished at a rate of 10 frames/second. As the hand-held ultrasound probe assembly 30 is translated from position 30d with corresponding ultrasound beam 50d and a corresponding ultrasound image to position 30i with corresponding ultrasound beam position 50i and a corresponding ultrasound image, there are 4 intermediate positions as seen by ultrasound beams 50e-50h.

The spacing between images in the scan (e.g. image-to-image spacing) may be used to detect that the imaging device is being rotated, translated, or moved on the target tissue. The image-to-image spacing may be determined by computing the maximum chord or distance, x between successive planar ultrasound scan frames at the maximum intended depth of ultrasound interrogation (i.e., maximum depth of the breast tissue in the present example). This maximum distance, x can be computed between the distal boundaries of each successive ultrasound scan frame (e.g., between ultrasound beam 50g and 50h, and corresponding images, since the position of the ultrasound transducer array 57 and/or the orientation of the hand-held ultrasound probe assembly 30 is precisely known at all time points when ultrasound scan frames are generated and recorded.

For the case of one embodiment involving the use of an Ascension Technologies position sensor product, the position of each sensor is determined (in one example version of a product sold by Ascension Technologies but not intended as a limitation as the data update rate may be higher or lower) at a rate of 120 times per second which is an order of magnitude more frequently than the repetition rate for ultrasound scan frames. As a consequence, the precise location of the ultrasound scan frame and, thereby, the precise location of the 240,000 pixels within each ultrasound scan frame, will be known in three-dimensional space as each ultrasound scan frame is generated by the ultrasound system 12 and recorded by the data acquisition and display module/controller 40. According, knowing the position of all pixels within each successive frame will enable the maximum distances between corresponding pixels in successive frames to be computed, focusing on those portions of successive ultrasound beams 50d-50h, and corresponding ultrasound images, that are known to be furthest apart, i.e., at locations within the recorded scan frame most distant from the ultrasound transducer array 57.

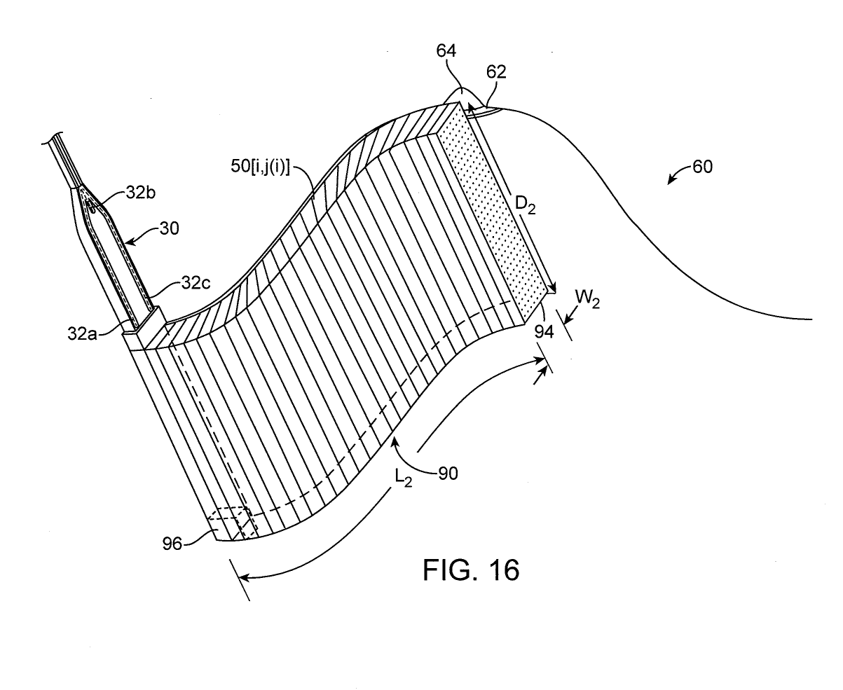

Referring now to FIG. 16, another algorithm for detecting movement of an imaging device (e.g. hand-held ultrasound probe assembly 30) is illustrated. This involves computation of the pixel density in each unit volume 96 within the swept volume 90 of the scan sequence, i containing N ultrasound beams 50[i,j(i)] and associated recorded frames where i equals the number of scan sequences and j(i) equals the number of emitted beams 50 and associated recorded frames for each scan sequence, i.

### Recording Based on Movement and Image Quality

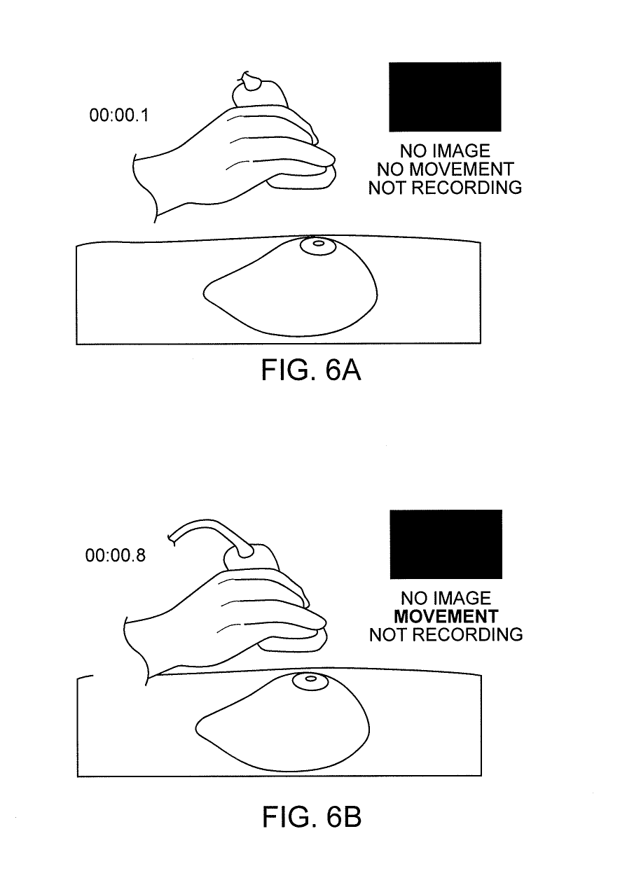

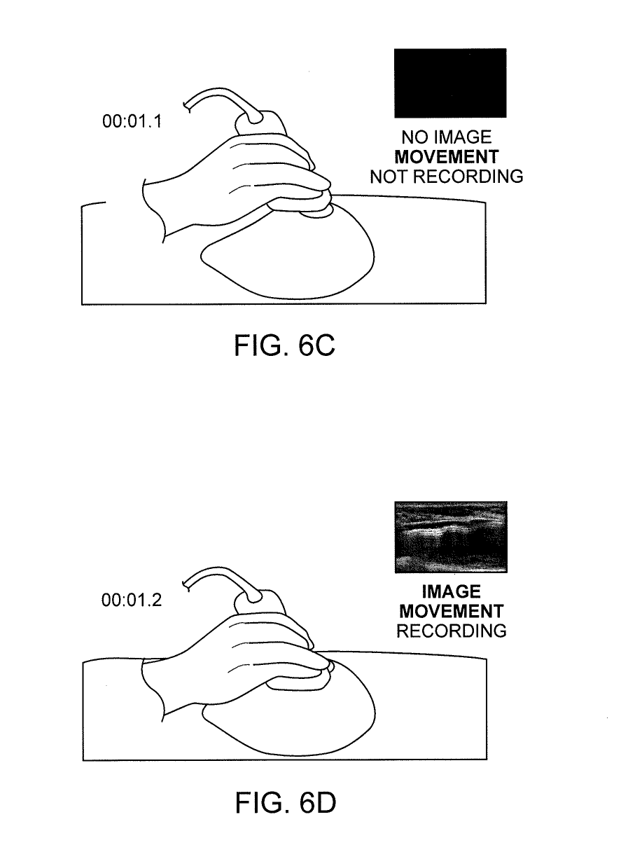

In some embodiments, by combining the factors of movement and image quality, parameters could be set up so that the system automatically records images without the need for active user intervention (such as a button, foot pedal, or voice command). In this scenario (see FIGS. 6-10), as long as the image has information and as long as there is “movement” between that image with information and the previous image (whether recorded or not), then that image is recorded.

FIGS. 6A-D show the automatic activation of the recording function once both movement and image quality criteria are satisfied. FIG. 6A shows the imaging device in a non-recording mode. No image is recorded by the image recording system from the imaging device. This is because image recording system does not detect any movement of the imaging device. Moreover, the image received by the image recording system from the imaging device is “black” without a pixel coloring pattern that corresponds to tissue imaging. As such, the pixel analysis of the received image does not activate recording.

FIGS. 6B-C shows movement by the imaging device but no recording by the image recording system. The automated recording system detects movement by the probe; however, image analysis (e.g. pixel analysis) indicates that the threshold amount of usable information has not been met.

FIG. 6D shows the recording of images by the image recording system. Once both movement and image quality criteria have been satisfied, the recording system automatically begins recording.

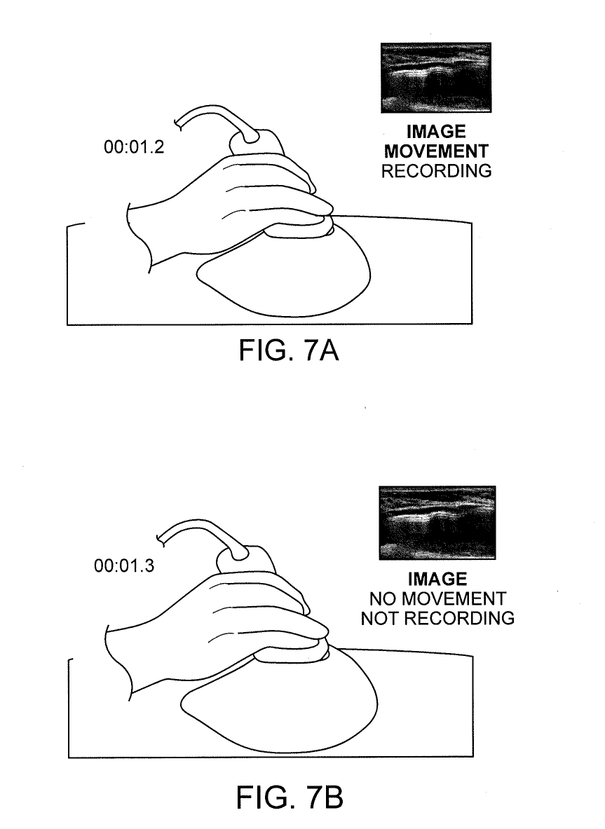

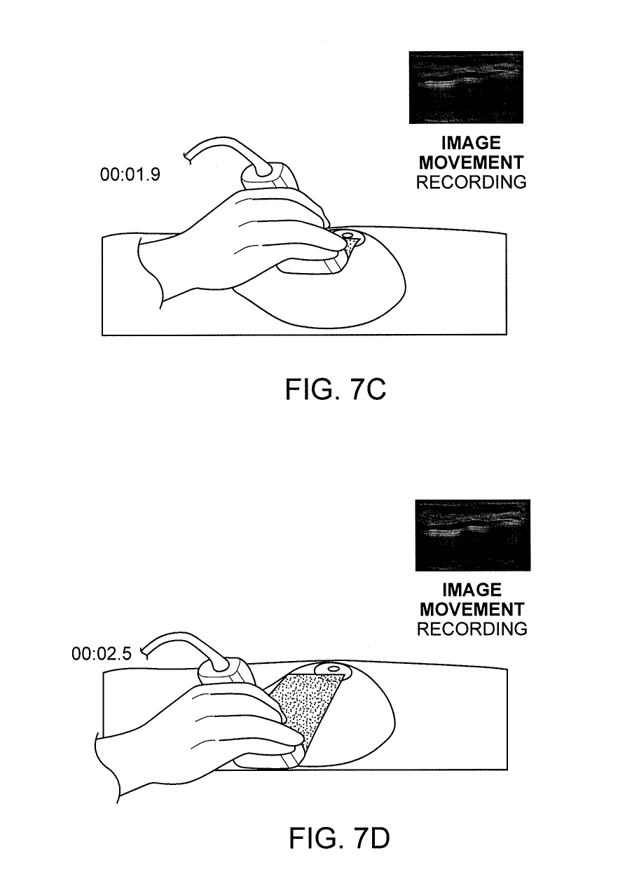

FIGS. 7A-D provide an example where the recording is stopped when image quality and movement criteria are not satisfied. As shown in FIG. 7B, a satisfactory image without movement results in no recording. FIG. 7A and FIG. 7C-D show the recording mode with both image quality and movement criteria satisfied.

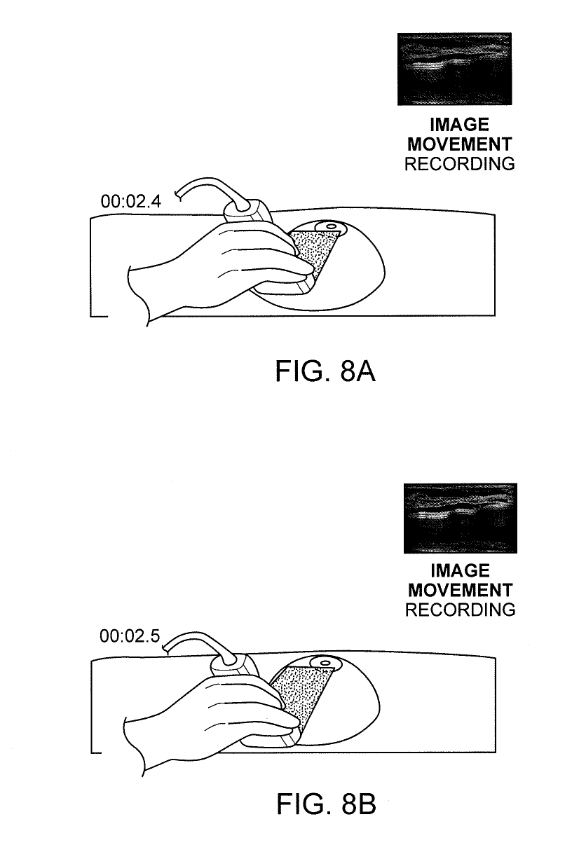

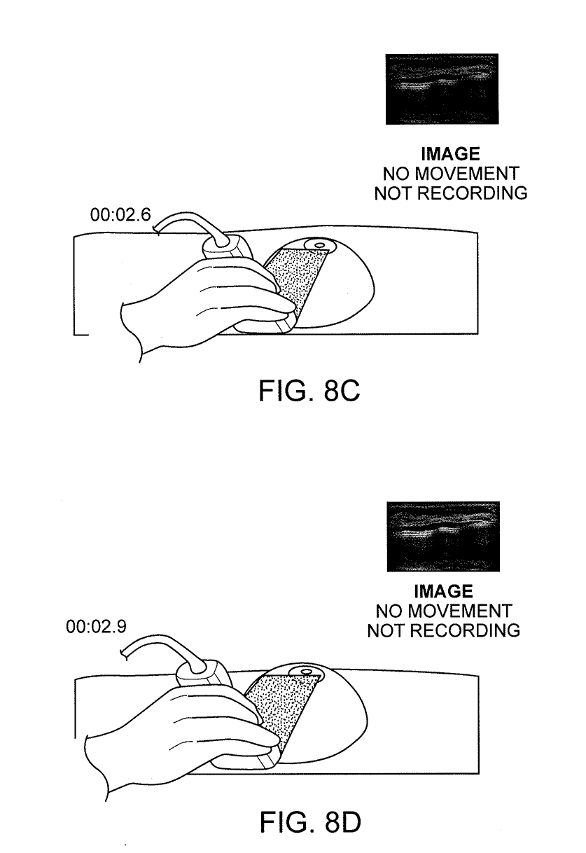

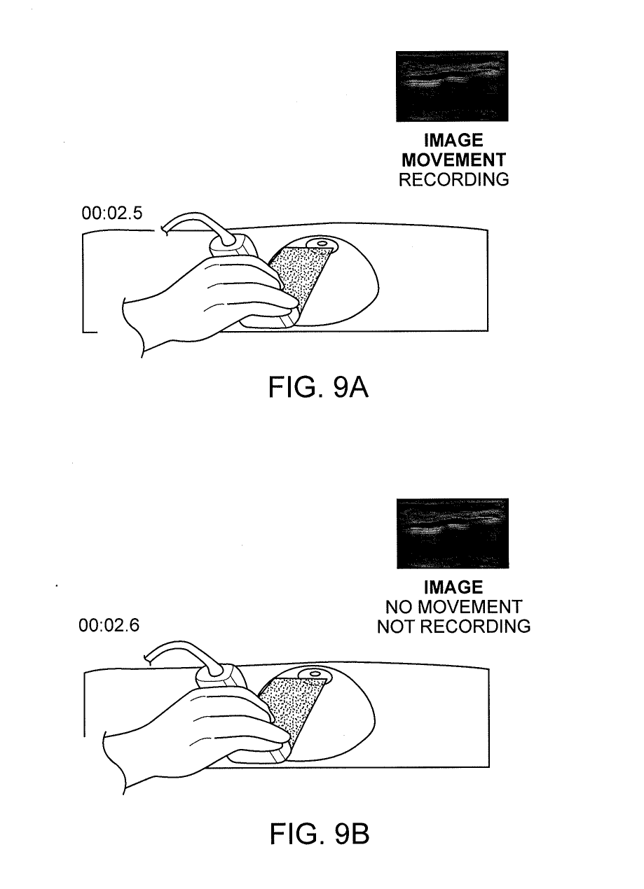

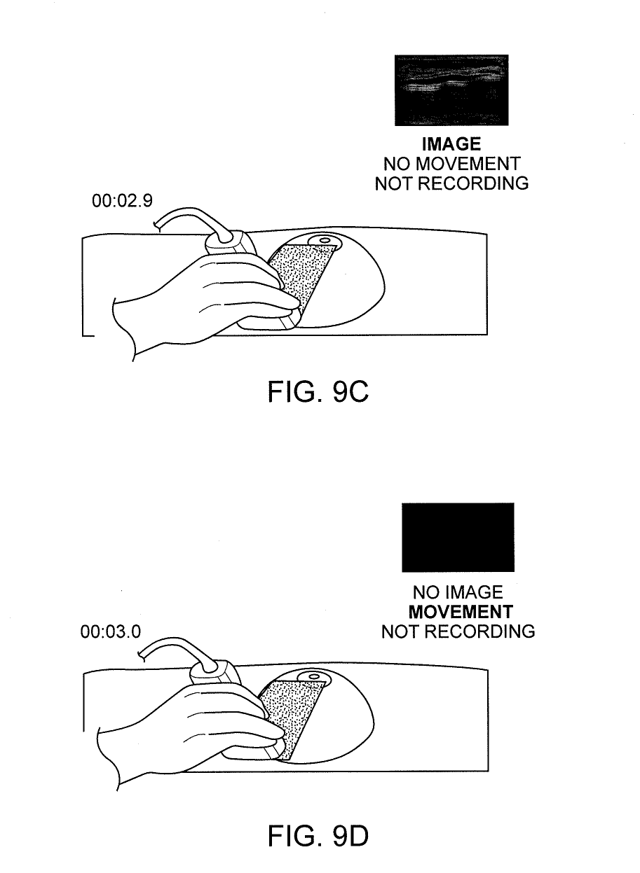

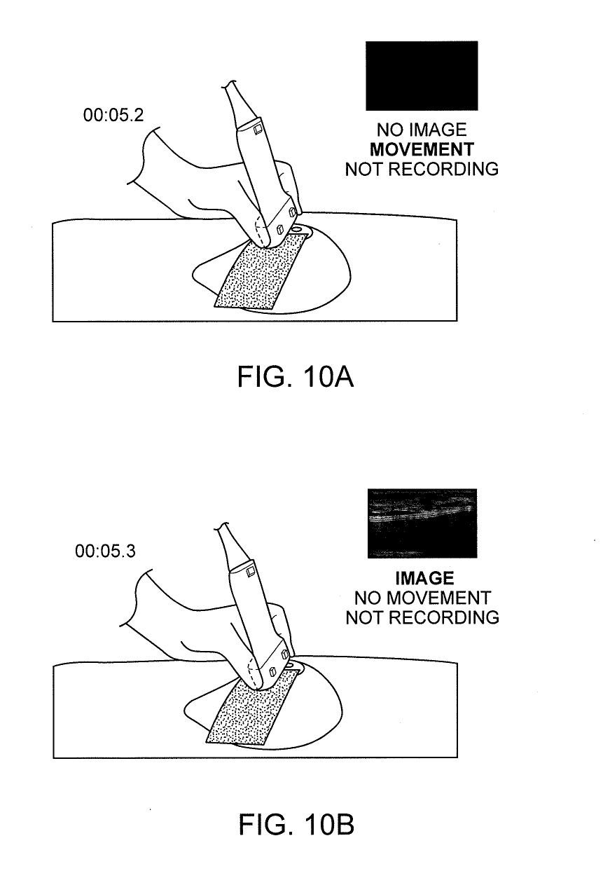

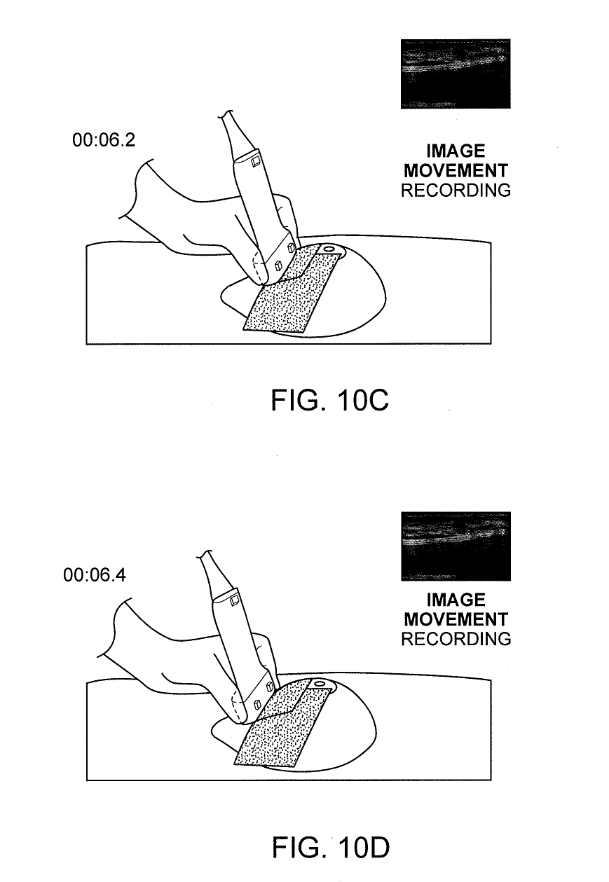

FIGS. 8A-D shows another example. Image quality and movement in FIGS. 8A-8B results in an image being recorded. But no image is recorded in FIGS. 8C-8D. FIGS. 9A-10D provide additional examples.

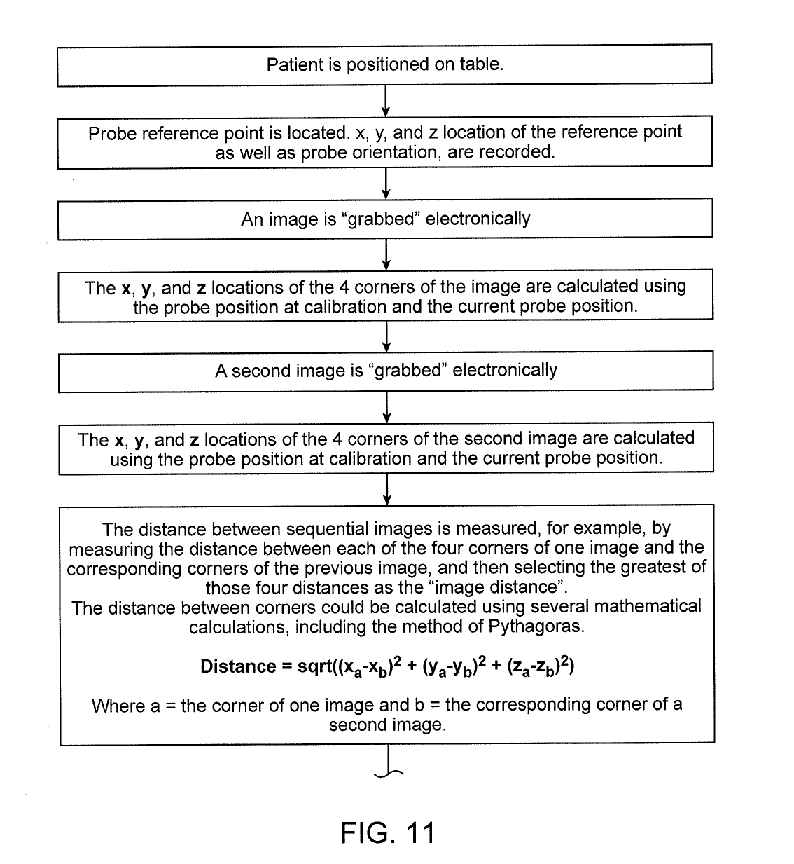

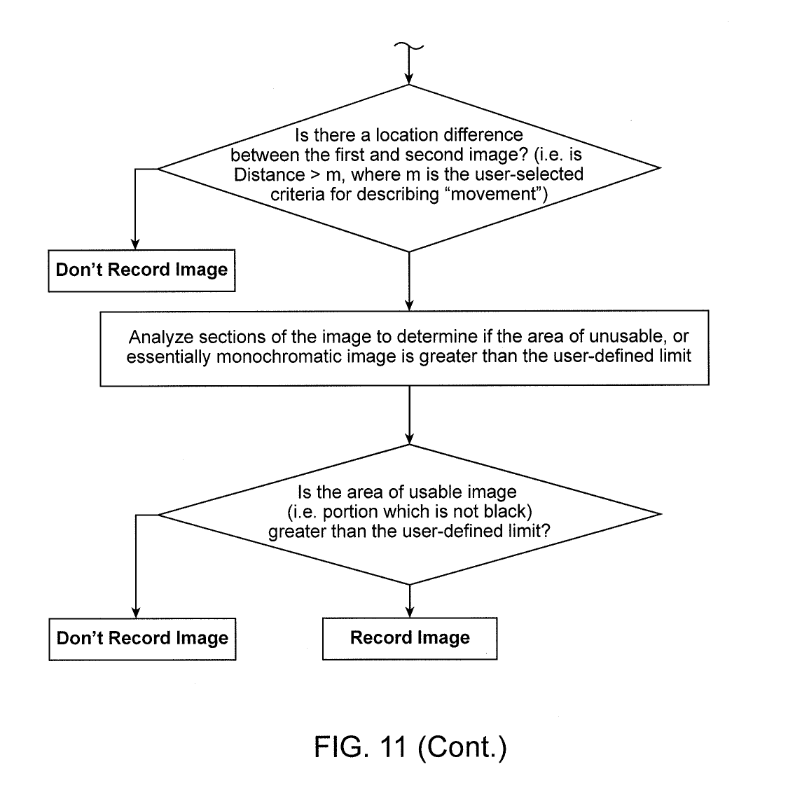

FIG. 11 illustrates an example of the recording process for systems described. A patient is positioned for the imaging procedure. In some embodiments, a reference point and other location data may be gathered for the patient. For example, the coronal, sagittal, and transverse planes for the patient may be determined. Additional details for methods and systems for gathering patient reference information (e.g. mapping information) are provided in U.S. Patent Application No.: 61/840,277 filed on Jun. 27, 2013, which is incorporated by reference herein in its entirety.

In some embodiments, referring to FIGS. 11 and 13, the automated imaging recording system 10 will electronically receive or grab images generated by the imaging device (e.g. ultrasound probe). Based on the position data received from the position tracking system 20, the recording system 10 can append location identifiers such as x, y, z values to the image. In the case of a planar rectangular or square image formed from a pixel array, the x,y,z values may be calculated for the corners or corner pixels. These steps are repeated for a second image captured or received from the imaging system 12.

The image-to-image distance between the first and second images is calculated. This may be performed by any mathematical method including the Pythagorean theorem.

If the distance between the first and second images does not satisfy a minimum distance, then the image(s) is not recorded. In some cases, the first image is recorded. A second image is not recorded until its distance from the previous meets a minimum standard. The next image is not recorded until its distance meets a minimum standard.

If the distance between the first and second images satisfies a minimum distance, then an image analysis action is performed. The recording system determines if an image contains sufficient usable information. Alternatively, the recording system can determine if an image contains an unacceptable amount of unusable information. In some cases, unusable information corresponds to monochromatic area(s). The recording system may compare the computed amount of unusable information in the image with a user defined limit or other preset value. If the image satisfies the image analysis criteria, then the image is recorded.

In some embodiments, the image recording system is configured to perform the image analysis and/or image device movement analysis as described above to determine whether an image is recorded. The image recording system may include computer software instructions or groups of instructions that cause a computer or processor to perform an action(s) and/or to make decisions. In some variations, the system may perform functions or actions such as by functionally equivalent circuits including an analog circuit, a digital signal processor circuit, an application specific integrated circuit (ASIC), or other logic device. In some embodiments, the image recording system includes a processor or controller that performs the functions or actions as described. The processor, controller, or computer may execute software or instructions for this purpose.

“Software”, as used herein, includes but is not limited to one or more computer readable and/or executable instructions that cause a computer or other electronic device to perform functions, actions, and/or behave in a desired manner. The instructions may be embodied in various forms such as objects, routines, algorithms, modules or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software may be dependent on, for example, requirements of a desired application, the environment it runs on, and/or the desires of a designer/programmer or the like. It will also be appreciated that computer-readable and/or executable instructions can be located in one logic and/or distributed between two or more communicating, co-operating, and/or parallel processing logics and thus can be loaded and/or executed in serial, parallel, massively parallel and other manners.

In some embodiments, the methods described may be performed by an imaging recording system that also performs additional other functions such as measuring coverage and resolution of images in a single and subsequent scan tracks and generating a tissue map.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

1. A tissue imaging system comprising:
an image recording system in communication with a manual imaging device having an imaging probe, the manual imaging device configured to scan a volume of tissue and output a first scan image and a second scan image, the image recording system configured to:
electronically receive the first and second scan images;
calculate an image-to-image spacing between the first and second scan images;
determine whether the image-to-image spacing indicates movement by the imaging probe;
determine an image quality of the first and second scan images if the image-to-image spacing indicates movement; and
record the first and second scan images where the calculated image-to-image spacing indicates movement by the imaging probe and the image quality analysis indicates that the first and second scan images satisfy a pre-determined image quality; and
a position tracking system configured to detect and track the position of the imaging probe and provide location identifier information for the first and second scan images, wherein the position tracking system electronically outputs probe position data and the location identifier information to the image recording system.

2. The system of claim 1, wherein the image recording system is configured to store a user-defined image-to-image distance limit for comparison to the calculated image-to-image spacing and automatically record the first and second scan images if the calculated image-to-image spacing is equal to or more than the user-defined image-to-image distance limit and if the scan images satisfy the pre-determined image quality.

3. The system of claim 2, wherein the user defined image-to-image spacing is about 1 mm or less.

4. The system in claim 1, further comprising electromagnetic position sensors.

5. The system in claim 1, further comprising magnetic position sensors.

6. The system of claim 1, further comprising microwave position sensors.

7. The system in claim 1, further comprising position sensors that are optical markers imaged by a plurality of cameras.

8. The system in claim 1, wherein the position sensors are infrared markers imaged by a plurality of cameras.

9. The system in claim 1, wherein the position sensors are ultraviolet markers imaged by a plurality of cameras.

10. The system of claim 1, wherein the image quality is determined based on a percentage of tissue imaged information present in the scan image.

11. The system of claim 10, wherein the pre-determined image quality is tissue imaged information present in the scan image of greater than about 50%.

12. The system of claim 10, wherein the percentage is a user-defined value corresponding to the variation in gray-scale within an image area of neighboring pixels.

13. The system of claim 12, wherein the user-defined value for the variation in the gray-scale within the image area corresponds to pixel value differences of less than 16 values on a 256 level gray scale.

14. The system of claim 1, wherein the system is configured to only record images that indicate movement of the imaging probe and satisfy the image quality analysis.

15. The system of claim 1, further comprising orientation sensors.

16. A method of recording images of a tissue structure comprising:
electronically receiving a first scan image generated from an imaging device;
electronically receiving a second scan image generated from an imaging device;
calculating an image-to-image spacing between the first and second scan images based on position data received from a plurality of sensors coupled to the imaging device;
comparing the calculated image-to-image spacing to a stored predetermined distance value;
performing an image quality analysis on the first and second scan images if the image-to-image spacing exceeds the stored predetermined distance value;
recording the first scan image if the first scan image satisfies the image quality analysis; and
recording the second scan image if the second scan image satisfies the image quality analysis.

17. The method of claim 16, wherein the image quality analysis is a pixel color analysis.

18. The method of claim 17, wherein the pixel color analysis includes grouping together neighboring pixels that are within 16 values on a 256 level gray scale of a user-defined value.

19. The method of claim 16, wherein the processor is configured to divide the first and second scan images into segments and compute the pixel values for each segment for image quality analysis.

20. The method of claim 16, wherein the imaging device is an ultrasound probe.

21. The method of claim 16, wherein the sensors comprise position sensors.

22. The method of claim 16, wherein the sensors comprise orientation sensors.

23. The method of claim 16, wherein the first and second scan images are only recorded if the image-to-image spacing exceeds the predetermined distance value and the scan images satisfy the image quality analysis.

24. The method of claim 16, wherein the image quality is determined based on a percentage of tissue imaged information present in the scan image.

25. The method of claim 24, wherein the pre-determined image quality is tissue imaged information present in the scan image of greater than about 50%.

26. The method of claim 16, wherein the user defined image-to-image spacing is about 1 mm or less.

27. A method of recording images of a tissue structure comprising:
electronically receiving a position data for an imaging probe from position and/or orientation sensors coupled to the probe to detect movement of the imaging probe;
electronically receiving a first scan image generated from the imaging probe;
performing an image quality analysis on the first scan image if movement of the imaging probe is detected; and
recording the first image if the first scan image satisfies the image quality analysis and movement of the imaging probe is detected.

28. The method of claim 27, wherein the movement of the probe is detected based on the position and/or orientation sensors coupled to the probe.

29. The method of claim 28, wherein movement is determined based on an image-to-image spacing computed by a processor and compared to a predetermined distance value stored in the processor.

30. The method of claim 29, wherein the predetermined distance value is about 1 mm or less.

31. The method of claim 27, wherein the image quality is determined based on a pre-determined percentage of tissue imaged information present in the scan image.

32. The system of claim 31, wherein the pre-determined image quality is tissue imaged information present in the scan image of greater than about 50%.

33. The method of claim 27, wherein the image quality analysis is a pixel color analysis.

34. The method of claim 33, wherein the pixel color analysis includes grouping together neighboring pixels that are within 16 values on a 256 level gray scale of a user-defined value.

35. A tissue imaging system comprising:
an image recording system in communication with a manual imaging device having an imaging probe, the manual imaging device configured to scan a volume of tissue and output a first scan image and a second scan image, the image recording system configured to:
electronically receive the first and second scan images;
calculate an image-to-image spacing between the first and second scan images;
determine whether the image-to-image spacing indicates movement by the imaging probe;
determine an image quality of the first and second scan images if the image-to-image spacing indicates movement; and
automatically record the first and second scan images where the calculated image-to-image spacing indicates movement by the imaging probe and the image quality analysis indicates that the first and second scan images satisfy a pre-determined image quality; and
a position tracking system configured to detect and track only the position or position and orientation of the imaging probe and provide location identifier information for the first and second scan images, wherein the position tracking system electronically outputs probe position data and the location identifier information to the image recording system.

36. The system of claim 35, further comprising position sensors or position and orientation sensors.

37. The system of claim 35, wherein the image recording system is configured to store a user-defined image-to-image distance limit for comparison to the calculated image-to-image spacing and automatically record the first and second scan images if the calculated image-to-image spacing is equal to or more than the user-defined image-to-image distance limit.

38. The system of claim 37, wherein the user defined image-to-image spacing is about 1 mm or less.

39. The system of claim 35, wherein the image quality is determined based on a percentage of tissue imaged information present in the scan image.

40. The system of claim 39, wherein the pre-determined image quality is tissue imaged information present in the scan image of greater than about 50%.

41. The system of claim 39, wherein the percentage is a user-defined value corresponding to the variation in gray-scale within an image area of neighboring pixels.

42. The system of claim 41, wherein the user-defined value for the variation in the gray-scale within the image area of neighboring pixels corresponds to pixel value differences of less than 16 values on a 256 level gray scale.

43. The system of claim 35, wherein the system is configured to only record images that indicate movement and satisfy the image quality analysis.
